(19) United States
(12) Patent Application Publication
(10) Pub. No.: US 2018/0179493 A1
(43) Pub. Date: Jun. 28, 2018

(54) MULTILAYER SHEET OF CELLS

(71) Applicant: The University of Manchester, Manchester (GB)

(72) Inventors: Brian DERBY, Manchester (GB); Rachel LENNON, Manchester (GB); Rachel SAUNDERS, Manchester (GB)

(73) Assignee: The University of Manchester, Manchester (GB)

(21) Appl. No.: 15/739,409

(22) PCT Filed: Jun. 24, 2016
(86) PCT No.: PCT/GB2016/051901

§ 371 Date: Dec. 22, 2017

(30) Foreign Application Priority Data
Jun. 25, 2015 (GB) 1511224.6

(51) Int. Cl. C12N 5/071; G01N 33/50; A61M 1/16

(52) U.S. Cl. CPC C12N 5/069; G01N 33/5014; G01N 33/5044; G01N 33/5064; C12N 2539/10; C12N 5/0697; A61M 1/1621; C12N 2502/28; C12N 2502/256; C12N 5/0686

(57) ABSTRACT

A multilayer sheet of cells, comprising a layer of endothelial cells, and a layer of podocytes is disclosed.

### FIELD OF THE INVENTION

The present invention relates to multilayer cell sheets, in particular to a multilayer sheet of cells comprising a layer of endothelial cells, and a layer of podocytes.

### BACKGROUND TO THE INVENTION

The kidney is a major site of organ damage caused by drug toxicity and this frequently manifests during drug development and/or in standard clinical care. Nephrotoxicity from drug exposure is estimated to contribute to 19-25% of all cases of acute kidney injury (AKI). Nephrotoxicity that leads to AKI is a major cause of drug attrition during pre-clinical, clinical and post approval stages of pharmaceutical development. Glomerular disease or damage can affect filtration of the blood which may lead to conditions such as proteinuria.

Drugs can exert their effects on various targets in the kidney and through a variety of cellular mechanisms which can be compounded by increase in concentration of the filtrate as it is processed by the kidney. The particular susceptibility of the kidney to drug toxicity can largely be attributed to its anatomy and function. The kidneys filter 150-180 litres of plasma per day and the filtrate can be concentrated from between threefold to in excess of one-hundred as it is processed. It is this increase in concentration can result in severe damage from nephrotoxic agents that may be present.

Some of the existing models for studying renal toxicity of drugs use monolayer cultures of a single kidney cell type, typically an epithelial cell type, e.g. in a transwell format. Byron et al. J Am Soc Nephrol (2014) 25: 953-966 describe an alternative arrangement in which glomerular endothelial cells and podocytes are co-cultured in vitro on tissue culture plates. These existing models are limited in complexity, and are unable to successfully mimic the complex structures found in the kidney. A consequence of this is that they are unable to reliably predict clinical outcomes.

The current limitations of preclinical in vitro and animal models that fail to reliably predict adverse nephrotoxic drug effects at an early enough point in the drug development timeline is apparent when the following statistics are considered. Only 2% of drug development failures due to nephrotoxicity occur in preclinical studies but this rises to 19% of failures in Phase 3 and post approval stages. Also, the nephrotoxic potential of newly approved drugs is often underestimated. Regulatory approved or validated in vitro models for the prediction of nephrotoxicity are currently not available.

There is a need in the art to provide model systems which more closely mimic the complex tissues of the kidney, in particular the renal corpuscle filtration barrier, for use to both predict and investigate drug-induced toxicities in the kidney, that can be applied reliably at an earlier stage of the drug discovery process to deliver more accurate and realistic toxicological predictions that closely correlate to clinical outcomes.

### SUMMARY OF THE INVENTION

The present invention provides a bioengineered (i.e. synthetic) renal corpuscle filtration barrier. The synthetic barrier has a composition and structure which mirrors the arrangement at the interface between glomerular capillaries and Bowman's capsule in the kidney. The synthetic barrier provides a physiologically accurate in vitro model, and is useful in a variety of applications, including renal toxicity studies for drug development.

In a first aspect, the present invention provides a multilayer sheet of cells, comprising a layer of endothelial cells, and a layer of podocytes.

In some embodiments, the endothelial cells are glomerular endothelial cells. In some embodiments, the multilayer sheet of cells further comprises an extracellular matrix disposed between the cell layers.

In some embodiments, the multilayer sheet of cells is provided on a support surface. In some embodiments, the multilayer sheet of cells is detachable from the support surface without enzymatic treatment, optionally wherein the support surface is a temperature responsive support surface.

In some embodiments, the cell layers are confluent cell layers.

In some embodiments, the multilayer sheet of cells is tolerant of flow conditions. In some embodiments, the multilayer sheet of cells is impervious to proteins larger than about 100 kDa.

In a related aspect, the present invention provides a bilayer sheet of cells, comprising, or consisting of, a layer of endothelial cells, and a layer of podocytes, optionally wherein the bilayer comprises, or consists of, a layer of endothelial cells, a layer of podocytes and extracellular matrix.

In another aspect, the present invention provides an in vitro cell culture, comprising a layer of endothelial cells, and a layer of podocytes, optionally wherein the endothelial cells are glomerular endothelial cells.

In some embodiments, the in vitro cell culture comprises an extracellular matrix disposed between the cell layers.

In some embodiments, the in vitro cell culture is provided on a support surface. In some embodiments, the support surface is a surface from which the multilayer sheet of cells is detachable without enzymatic treatment, optionally wherein the support surface is a temperature responsive support surface.

In some embodiments, the cell layers of the in vitro cell culture are confluent cell layers.

In another aspect, the present invention provides a device, having two compartments separated by a multilayer sheet of cells according to the present invention.

In some embodiments, the device additionally comprises a liquid/fluid permeable support for the multilayer sheet of cells. In some embodiments, the device comprises a liquid/fluid inlet to the compartment adjacent the layer of endothelial cells, a liquid/fluid outlet from the compartment adjacent the layer of endothelial cells, and a liquid/fluid outlet from the compartment adjacent the layer of podocytes.

In some embodiments, the device additionally comprises a compartment comprising renal epithelial cells. In some embodiments, the compartment comprising renal epithelial cells is in liquid/fluid communication with the compartment adjacent the layer of podocytes.

In another aspect, the present invention provides the use of a multilayer sheet of cells according to the invention, or of a device according to the present invention, in an in vitro assay.

In some embodiments the assay is a toxicity assay. In some embodiments, the assay measures transfer of a substance across the multilayer sheet of cells. In some embodiments, the assay measures a property of the multilayer sheet of cells, or a property of a component of the multilayer sheet of cells.

In another aspect, the present invention provides an artificial kidney comprising a multilayer sheet of cells according to the present invention, or a device according the present invention.

In another aspect, the present invention provides a dialysis apparatus, comprising a multilayer sheet of cells according to the present invention, or a device according the present invention.

In another aspect, the present invention provides a multilayer sheet of cells, a device, an artificial kidney or dialysis apparatus according to the present invention, to filter blood or plasma.

In another aspect, a multilayer sheet of cells, a device, an artificial kidney or dialysis apparatus according to the present invention is provided for use in a method of treatment by dialysis.

In another aspect, the present invention provides a method of treating a patient in need of dialysis, the method comprising use of a multilayer sheet of cells, a device, an artificial kidney or dialysis apparatus according to the present invention, to dialyse blood or plasma.

In another aspect, the present invention provides a method for producing a multilayer sheet of cells, the method comprising: (i) providing a layer of endothelial cells; (ii) providing a layer of podocytes; and (iii) contacting the layers of cells and culturing the contacted cell layers in vitro; thereby producing a multilayer sheet of cells.

In another aspect, the present invention provides a method for producing a multilayer sheet of cells, the method comprising: (i) providing a layer of endothelial cells, optionally glomerular endothelial cells; (ii) layering podocytes onto the layer of endothelial cells; and (iii) culturing the cells in vitro; thereby producing a multilayer sheet of cells.

In some embodiments, step (i) comprises contacting endothelial cells or glomerular endothelial cells with a support surface, and culturing the cells on the support surface in vitro.

In some embodiments, the support surface is a surface from which the multilayer sheet of cells is detachable without enzymatic treatment, optionally wherein the support surface is a temperature responsive support surface.

In some embodiments, the method additionally comprises: (iv) detaching the multilayer sheet of cells from the support surface.

In another aspect, the present invention provides a method for producing a multilayer sheet of cells, the method comprising: (i) providing a layer of podocyte cells; (ii) providing a layer of endothelial cells, optionally glomerular endothelial cells, (iii) placing the layer of endothelial cells onto the layer of podocyte cells, or placing the layer of podocyte cells onto the layer of endothelial cells; and (iii) culturing the cells in vitro; thereby producing a multilayer sheet of cells. In some embodiments one or both of the layer of podocyte cells of (i) and layer of endothelial cells of (ii) is a layer on a support surface from which the cell layer is detachable without enzymatic treatment, optionally wherein the support surface is a temperature responsive support surface. The method may additionally comprise detaching the multilayer sheet of cells from the support surface.

In another aspect of the present invention, a bilayer sheet of cells consisting of a layer of endothelial cells and a layer of podocytes is provided.

In another aspect of the present invention, a bilayer sheet of cells consisting of a layer of endothelial cells, a layer of podocytes and extracellular matrix disposed between the cell layers.

### DESCRIPTION

### Renal Corpuscle Filtration Barrier

In the kidney, blood filtration takes place at the renal corpuscle, which consists of a glomerulus surrounded by a Bowman's capsule. The structure of the renal corpuscle filtration barrier is reviewed, for example, in Miner et al., Pediatr Nephrol (2011) 26(9): 1413-1417, which is hereby incorporated by reference in its entirety.

A glomerulus is a network of capillaries. The capillaries of the glomerulus contain endothelial cells and mesangial cells. The endothelial cells of the glomerular capillaries are fenestrated. The mesangial cells are modified smooth muscle cells that lie between the capillaries, and function to regulate blood flow. The capillaries of the glomerulus receive blood from an afferent arteriole, and the blood drains to an efferent arteriole. Both arterioles are high resistance, resulting in high pressure in the glomerulus which facilitates ultrafiltration of water and small molecules into the Bowman's capsule.

The Bowman's capsule has an outer parietal layer of simple squamous epithelium, and an inner visceral layer of squamous epithelium lined by podocytes. Podocytes have projections called pedicels, which wrap around glomerular capillaries. Pedicels of adjacent podocytes interdigitate to form filtration slits. Filtration slits are covered by slit diaphragms which are modified tight junctions, comprising e.g. nephrin, podocalyxin and P-cadherin, which restrict passage of large proteins into the space of the Bowman's capsule.

Between the layer of podocytes and the layer of fenestrated endothelial cells there is a glomerular basement membrane (GBM). The glomerular basement membrane is composed of three layers: (i) the lamina rara externa, adjacent to the podocytes, (ii) the lamina densa, a central layer comprising type IV collagen and laminin, and which acts as a filter to prevent the passage of large proteins, and (iii) the lamina rara interna, adjacent to the endothelial cells. The lamina rara externa and the lamina rara interna contain heparan sulfate, a negatively charged glycosaminoglycan which contributes to preventing the passage of negatively charged molecules.

The renal corpuscle filtration barrier comprises the fenestrated endothelial cells of the glomerular capillaries, the glomerular basement membrane, and the filtration slits of the podocytes. The barrier permits passage of water, ions, and small molecules (e.g. salts) from the bloodstream into the space of the Bowman's capsule (that is, the space between the visceral and parietal layers), but does not allow passage of large proteins and negatively-charged molecules, which are retained in the blood. Filtrate entering the Bowman's capsule empties into the proximal tubule of the nephron.

The present invention provides what is essentially a synthetic renal corpuscle filtration barrier prepared in vitro by the controlled coculture of endothelial cells and podocytes. The renal corpuscle filtration barrier of the present invention is therefore artificial, i.e. non-natural.

### A Multilayer Sheet of Cells

In a first aspect, the present invention provides a multilayer sheet of cells, comprising a layer of endothelial cells, and a layer of podocytes.

An endothelial cell layer is preferably adjacent a podocytes layer, optionally having a layer of extracellular matrix or basement membrane therebetween.

A multilayer sheet of cells as used herein refers to a sheet of cells which comprises more than one layer of cells, e.g. two or more layers of cells. In some embodiments, a multilayer sheet of cells may be a bilayer sheet of cells (i.e. having two layers of cells). In some embodiments, a multilayer sheet of cells may comprise more than two layers cells, e.g. 3, 4, or 5 layers of cells. A multilayer sheet may preferably have an even number of layers of alternating endothelial cell layers and podocyte layers.

A layer of cells as used herein refers to a plurality of cells which are arranged substantially side-by-side, for example adhered to the same surface. Cells provided in a layer of cells may contact one another, and may, for example, form junctions between the cells.

Each individual layer of cells of the multilayer of cells of the present invention may independently be a monolayer of cells. That, is a layer of cells may be one cell thick.

In some embodiments, one or more of the layer of endothelial cells or layer of podocytes may be a confluent cell layer. In some embodiments, both the layer of endothelial cells and the layer of podocytes are confluent cell layers.

Confluence refers to the proportion of the area of a surface to which a layer of adherent cells is covered by the cells. By way of example, 50% confluence means that approximately half of the surface is covered by cells. A confluent layer of cells refers to a layer having about 100% confluence. That is, substantially all of the surface upon which the layer of cells is growing is covered by the layer of cells.

In some embodiments, one or both of the layer of endothelial cells and layer of podocytes of a multilayer sheet of the invention may have a confluence of one of about 70-100%, 80-100%, 90-100%, or 100% confluence.

In embodiments of the present invention, a layer of cells may be substantially of a single cell type. For example, a layer of endothelial cells may be substantially only of endothelial cells. Similarly, a layer of podocytes may be substantially only of podocytes.

A layer of endothelial cells may be substantially free of podocytes. Similarly, a layer of podocytes may be substantially free of endothelial cells.

It will be appreciated that the arrangement of cells in layers is distinct from a co-culture of cells. In a co-culture of different cell types cells of different types may be mixed in one layer. In some embodiments of the present invention, a single layer of cells which comprises both endothelial cells and podocytes is optionally excluded.

A sheet as used herein refers to an arrangement of two of more layers of cells, one on top of another. As such, the sheet is formed by adjacent layers of cells, one layer on top of the other, each layer being substantially of one cell type.

Layers of adjacent cells are therefore preferably in contact with one another. Where an extracellular matrix or basement membrane is present the cells may be separated only by that extracellular matrix or basement membrane, and even in the presence of the extracellular matrix or basement membrane the cells in adjacent layers may still be in contact with each other. The extracellular matrix or basement membrane is preferably the product of one or both cells layers, e.g. formed by proteins, glycosaminoglycans and proteoglycans secreted by cells in one or both adjacent layers.

Adjacent layers of cells are therefore preferably not separated by a support surface, e.g. plastic, mesh, web, cell culture surface, membrane, semi-permeable membrane, nanofiber membrane, nickel support mesh, PCL layer, or polycarbonate layer. Adjacent layers of cells are preferably not separated by an exogenously added layer of material, e.g. layer of extracellular matrix, layer of gel, layer of collagen (e.g. collagen gel), fibrin, fibronectin, laminin, vitronectin, entactin or mixture of such materials, e.g. Matrigel™.

In a layer of cells which is substantially of one cell type, 70%, 80%, 90%, 95% or 99% or more of the cells in the layer of cells may be of the same cell type.

A sheet of cells may be provided in a substantially planar arrangement, e.g. as a flat sheet. In some embodiments, the multilayer sheet of cells may be formed to a three dimensional structure, e.g. using a mould. In some embodiments, the multilayer sheet of cells is flexible, and/or can be rolled or folded.

The multilayer sheet of cells of the present invention may be an isolated multilayer sheet of cells. In some embodiments, the multilayer sheet of cells may be physically separate from other structures, for example, the multilayer sheet of cells may be provided as an independent and self-contained product.

In some embodiments, the multilayer sheet of cells may additionally comprise culture media, e.g. liquid or gel culture media containing nutrients and/or growth factors, preferably in contact with the sheet of cells. In some embodiments, the cell culture medium is a cell culture medium in which the endothelial cells and podocytes are viable. In some embodiments, the cell culture medium is Endothelial Basal Medium-2 (EBM-2; Lonza).

In some embodiments, the multilayer sheet of cells may be frozen, e.g. at about −80° C. or at a lower temperature below, at or just above the boiling point of liquid nitrogen (about −196° C.).

In some embodiments a multilayer sheet of cells according to the invention is transported as a live cell system. In some embodiments, the multilayer sheet of cells may be transported frozen, e.g. at about the temperatures described above.

In some embodiments, the multilayer sheet of cells may be transported as frozen cellular components (e.g. in vials), optionally with instructions for culture and assembly of the components to a multilayer sheet of cells according to the invention. That is, in some embodiments, the multilayer sheet of cells of the invention may be shipped as a vial of endothelial cells and a vial of podocytes.

The multilayer sheet of cells is not a naturally occurring product, being the product of manipulation and/or culture of cells in vitro.

### Cells

The multilayer sheet of cells and in vitro cell cultures of the present invention comprise a layer of endothelial cells, and an adjacent layer of podocytes.

In some aspects of the present invention, renal epithelial cells are provided.

The cells according to the invention can be from any suitable source for producing a multilayer sheet of cells according to the invention. For example, the cells may be mammalian cells, optionally non-human mammalian cells. The cells may be human, or may be non-human, e.g. from rabbit, guinea pig, rat, mouse or other rodent (including cells from any animal in the order Rodentia), cat, dog, pig, sheep, goat, cattle, horse, non-human primate or other non-human vertebrate organism. In particular embodiments, the cells are of human origin.

Cells may be primary cells, e.g. from a donor animal, or immortalised cell lines, e.g. available from cell depositary instututions such as ATCC.

“Expression” as used herein may be gene expression or protein expression. Assays for investigating gene or protein expression are well known to the skilled person. Gene expression may be investigated e.g. by reverse transcription-PCR (RT-PCR). Protein expression may be investigated e.g. by immunoassay, such as western blot, immunostaining and microscopy, or flow cytometry.

Cell morphology may be investigated using any suitable means. For example, morphology can be investigated by light microscopy or electron microscopy.

“Flow conditions” as used herein relates to conditions wherein liquid/fluid is flowed past the cells, or layer of cells, or multilayer sheet of cells. The liquid/fluid may, for example, be cell culture media, buffer, saline, or bodily fluid. A bodily fluid may for example be a fluid which is, or which is derived from, a quantity of blood, plasma, or serum.

“Plasma” as used herein refers to the liquid component of blood, lacking the cellular component, and may be the fluid portion of the blood obtained after removal of the blood cells. “Serum” used herein refers to plasma lacking clotting factors (e.g. fibrinogens), and may be the fluid portion of the blood obtained after removal of the fibrin clot and blood cells.

Cells, layers of cells or multilayer sheets of cells may be subjected to flow conditions in, for example, a flow chamber such as a QuasiVivo chamber (Kirkstall Ltd, UK). The flow rate in a chamber may be one of 300 to 800, 350 to 700, or 400 to 600 μl/min. In some embodiments, flow rate in a chamber may be about 540 μl/min 480 μl/min, 450 μl/min or 430 μl/min. Cells, layers of cells or multilayer sheets of cells may be subjected to flow conditions for one of about 1-14 days, 2-12 days, 3-10 days, 4-7 days, and about 5 days.

In some embodiments, one or more cells of the multilayer sheets of cells of the present invention may have modified expression of one or more molecules. Multilayer sheets of cells comprising cells having modified expression of one or more molecules are useful to study the structure and function of the renal corpuscle filtration barrier, and/or renal disease, as described herein below.

For example, in some embodiments, one or more cells of the multilayer sheets of cells of the present invention may have modified expression of one or more genes or proteins which are normally expressed by that cell type. In some embodiments, the gene or protein may be a gene or protein whose expression or activity is a marker for that cell type. In some embodiments expression of that gene or protein may be specific to that cell type.

In some embodiments, the multilayer sheet of cells may comprise endothelial cells having modified gene or protein expression of one or more of: platelet endothelial cell adhesion molecule 1 (PECAM1), intercellular adhesion molecule 2 (ICAM2), vascular endothelial cadherin (CD144), VEGF receptor 2 (VEGFR2), von Willebrand factor (vWF), Tie2, and E-selectin. In some embodiments, the multilayer sheet of cells may comprise podocytes having modified gene or protein expression of one or more of synaptopodin, nephrin, podocin, P-cadherin, and CD2AP.

### Endothelial Cells

The multilayer sheet of cells of the present invention comprises a layer of endothelial cells.

Any endothelial cells useful in producing a multilayer sheet according to the invention are contemplated.

The endothelial cells are preferably viable in culture with podocytes. In some embodiments, the endothelial cells are vascular endothelial cells. In some embodiments, the endothelial cells are fenestrated endothelial cells. In some embodiments, the endothelial cells are derived from fenestrated capillaries. In some embodiments, the epithelial cells are fenestrated, but lack diaphragms spanning the fenestrae. In some embodiments, the endothelial cells have fenestrae of diameter of about 50-400 nm, 50-120 nm, about 60-120 nm, or about 70-100 nm.

In particular embodiments, the endothelial cells are derived from kidney tissue. In some embodiments, the endothelial cells are glomerular endothelial cells. That is to say, the endothelial cells are derived from the capillaries of the glomerulus. In some embodiments, the cells are derived from isolated glomeruli. In some embodiments, the cells may be ACBRI 128 primary human glomerular microvascular endothelial cells (Cell Systems), or human renal glomerular endothelial cells (HRGEC; 3H Biomedical AB).

In some embodiments, the endothelial cells may be primary cells. In some embodiments, the endothelial cells may be immortalized or conditionally immortalized endothelial cells. Conditionally immortalized glomerular endothelial cells and methods for their preparation are described, for example, in Satchell et al., Kidney International (2006) 69, 1633-1640, which is hereby incorporated by reference in its entirety.

In some embodiments, the endothelial cells of the present invention may display particular characteristics or express particular markers. For example, the endothelial cells may display one or more of: expression of platelet endothelial cell adhesion molecule 1 (PECAM1), expression of intercellular adhesion molecule 2 (ICAM2), expression of vascular endothelial cadherin (CD144), expression of VEGF receptor 2 (VEGFR2), expression of von Willebrand factor (vWF), expression of Tie2, and expression of E-selectin in response to TNFα. In some embodiments, the endothelial cells may express PECAM1, ICAM2, VEGFR2 and vWF.

In some embodiments, the endothelial cells may have elongate morphology. In embodiments where the endothelial cells are exposed to flow conditions, the endothelial cells may be elongate in the direction of flow. In some embodiments, actin filaments of endothelial cells cultured under flow conditions may be shorter than actin filaments of endothelial cells cultured in the absence of flow conditions. In some embodiments, endothelial cells cultured under flow conditions may have actin filaments with a radial or star-shaped arrangement. In some embodiments, PECAM expression by endothelial cells cultured under flow conditions may be patch-like, as compared to a long and thin pattern of expression by endothelial cells cultured in the absence of flow conditions.

### Podocytes

The multilayer sheet of cells of the present invention also comprises a layer of podocytes.

The podocytes are preferably viable in culture with glomerular epithelial cells. In some embodiments, the podocytes are derived from kidney tissue. In some embodiments, the podocytes may be primary cells. In some embodiments, the podocytes may be immortalized or conditionally immortalized podocytes. Conditionally immortalized podocytes and methods for their preparation are described, for example, in Saleem et al., J Am Soc Nephrol (2002) 13, 630-638, which is hereby incorporated by reference in its entirety.

In some embodiments, the podocytes of the present invention may display particular characteristics or express particular markers. For example, the podocytes may express one or more of synaptopodin, nephrin, podocin, P-cadherin, and CD2AP.

In some embodiments, the podocytes may have processes. In some embodiments, processes of adjacent podocytes in a layer of podocytes may interdigitate. In some embodiments, the podocytes form filtration slits. In some embodiments, the filtration slits may have a diameter of one of 5-100 nm, 10-75 nm, 15-50 nm and 20-30 nm.

In some embodiments, adjacent podocytes in a layer of podocytes may comprise a filtration slit diaphragm between the podocytes. In some embodiments, the slit diaphragm may comprise one or more of ZO-1, α-catenin, β-catenin, γ-catenin and P-cadherin.

Where the podocytes are exposed to flow conditions, more focal adhesions may be formed by the podocytes, or the focal adhesions may be larger, as compared to the focal adhesions formed by the podocytes in the absence of flow conditions (e.g. static culture conditions). In some embodiments, localisation of vinculin of podocytes cultured under flow conditions may be primarily to focal adhesions.

In some embodiments, podocytes exposed to flow conditions may be more confluent or may reach confluence more quickly than podocytes cultured in the absence of flow conditions. In some embodiments, podocytes cultured under flow conditions may express more nephrin as compared to podocytes cultured in the absence of flow conditions. In some embodiments, actin filaments of podocytes cultured under flow conditions may be shorter than actin filaments of podocytes cultured in the absence of flow conditions. In some embodiments, the actin filaments of podocytes cultured under flow conditions may have a radial or star-shaped arrangement. In some embodiments, the cell surface of podocytes may be smoother following culture under static conditions as compared to culture under flow conditions. In some embodiments, podocytes cultured under flow conditions may have concentrations of actin fibres.

### Renal Epithelial Cells

In some aspects of the present invention, renal epithelial cells are provided. In some embodiments, the renal epithelial cells are tubule epithelial cells.

In some embodiments, the epithelial cells are normal rat kidney epithelial cells (NRK), human renal epithelial (hRE) cells, HK-2 cells, or human renal proximal tubule epithelial cells (hRPTECs). In some embodiments, the epithelial cells are Primary Renal Proximal Tubule Epithelial Cells (ATCC® PCS-400-010™), Primary Renal Cortical Epithelial Cells (ATCC® PCS-400-011™), or Primary Renal Mixed Epithelial Cells (ATCC® PCS-400-012™).

The luminal surface of the proximal tubule of the nephron, into which filtrate from renal corpuscle filtration enters, is lined with epithelial cells. The epithelia are provided with microvilli forming a brush border, which provides a large surface area for the resorption of e.g. ions and water from the filtrate.

In some embodiments, the renal epithelial cells are tubule epithelial cells. In some embodiments, the tubule epithelial cells are proximal tubule epithelial cells. In some embodiments, the renal epithelial cells are derived from kidney tissue.

In some embodiments, the renal epithelial cells may be primary cells. In some embodiments, the renal epithelial cells may be immortalized or conditionally immortalized cells. Conditionally immortalized tubule epithelial cells and methods for their preparation are described, for example, in Wilmer et al., Cell Tissue Res (2010) 339, 449-457, which is hereby incorporated by reference in its entirety.

In some embodiments, the renal epithelial cells may display particular characteristics or express particular markers. For example, the renal epithelial cells may express one or more of aminopeptidase N, zona occludens 1, aquaporin 1, dipeptidyl peptidase IV, multidrug resistance protein 4 and alkaline phosphatase.

### Glomerular Basement Membrane

In embodiments of the present invention, the multilayer sheet of cells comprises an extracellular matrix disposed between the layer of endothelial cells and the layer of podocytes.

Essentially, the extracellular matrix disposed between the layer of endothelial cells and the layer of podocytes is a glomerular basement membrane, or a pseudo glomerular basement membrane (i.e. a mimic of the glomerular basement membrane present in vivo.

In some embodiments, the extracellular matrix (ECM) comprises molecules secreted by the endothelial cells and/or podocytes.

In some embodiments, the extracellular matrix has a composition and/or structure similar to the glomerular basement membrane. The structure of the glomerular basement membrane is described, for example, in Farquar, J Clin Invest (2006) 116:2090-2093, which is hereby incorporated by reference in its entirety. The composition of the glomerular basement membrane and methods for determining the same, are described in, Lennon et al., J Am Soc Nephrol (2014) 25: 939-951, which is hereby incorporated by reference in its entirety.

In some embodiments, the multilayer sheet of cells comprises an extracellular matrix disposed between the layers of cells which comprises one or more of collagen IV, laminin, nidogen-1, nidogen-2, agrin, perlecan, and collagen XVIII. In some embodiments, the extracellular matrix comprises laminin, collagen IV, nidogens and heparan sulphate proteoglycan.

In some embodiments, the extracellular matrix has a thickness of one of about 50-700 nm, 100-600 nm, 150-550 nm, or 200-500 nm. Thickness of the extracellular matrix may be investigated e.g. by light microscopy or electron microscopy.

The extracellular matrix may be impervious to certain molecules. In some embodiments, the extracellular matrix may be impervious to proteins having a size larger than one of about 70 kDa, 80 kDa, 90 kDa or 100 kDa.

In some embodiments, the extracellular matrix allows passage of one or more of small molecules, water and ions. For example, in some embodiments, the extracellular matrix allows passage of one or more of urea, creatinine, or cystatin C.

In some embodiments, the extracellular matrix (a) is impervious to proteins having a size larger than one of about 70 kDa, 80 kDa, 90 kDa or 100 kDa, and (b) allows passage of one or more of small molecules, water and ions, e.g. urea, creatinine, and/or cystatin C.

### Properties of the Multilayer Sheet of Cells

Properties of constituents of the multilayer sheet of cells according to the invention are described hereinabove. The following section describes particular properties of the multilayer sheet of cells according to the present invention.

The multilayer sheet of cells of the present invention may be defined by reference to one or more structural or functional properties.

In some embodiments, the multilayer sheet of cells is impervious to large proteins.

That is to say, in some embodiments, the multilayer sheet of cells does not permit passage through the sheet—i.e. from the side of the sheet adjacent the endothelial cell layer to the side of the sheet adjacent the podocyte cell layer, or vice versa—of large proteins. For example, in some embodiments, the multilayer sheet of cells is impervious to proteins having a size larger than about one of 70 kDa, 80 kDa, 90 kDa or 100 kDa.

The integrity of the barrier provided by the multilayer sheet of cells can also be investigated by analysing the rate of exchange of molecules capable of passing from one side of the sheet to the other. For example, the rate of exchange of e.g. labelled albumin, e.g. human serum albumin could be investigated.

In some embodiments, the multilayer sheet of cells allows passage of one or more of small molecules, water and ions. For example, in some embodiments, the multilayer sheet of cells allows passage of one or more of urea, creatinine, or cystatin C.

In some embodiments, the multilayer sheet of cells (a) is impervious to proteins having a size larger than one of about 70 kDa, 80 kDa, 90 kDa or 100 kDa. and (b) allows passage of one or more of small molecules, water and ions, e.g. urea, creatinine, and/or cystatin C.

Whether a multilayer sheet of cells is impervious to a given molecule, or allows passage of a given molecule, can be determined by, for example, providing a labelled molecule to one side of the sheet of cells, and after an appropriate period of time and under suitable conditions, attempting to detect the label and/or labelled molecule on the other side of the sheet. For example, labelled (e.g. fluorescently labelled) protein may be provided to the side of the sheet adjacent the endothelial cell layer, and whether the label and/or labelled protein is detectable at the side of the sheet adjacent the podocyte cell layer may be investigated.

In some embodiments, the multilayer sheet of cells of the invention is tolerant of flow conditions. Flow conditions are defined hereinabove.

A multilayer sheet of cells which is tolerant of flow conditions may be a multilayer sheet of cells which maintains substantially the same structure and/or function under flow conditions as compared to structure and/or function when the multilayer sheet of cells is not under flow conditions, e.g. when the multilayer sheet of cells is in in vitro static culture.

Whether a multilayer sheet of cells is tolerant of flow conditions can be determined by providing the multilayer sheet of cells to a QuasiVivo chamber, providing flow conditions to the multilayer sheet of cells, and investigating one or more of (or a change in one or more of): cell morphology, cell death, cell viability, gene or protein expression by cells, confluence of cell layers, thickness of extracellular matrix between the layers of cells, and transfer of molecules across the multilayer sheet of cells.

Cell morphology may be investigated by reference to the properties of endothelial cells and podocytes of the multilayer sheet of cells of the present invention as described hereinabove. Confluence of cell layers and thickness of extracellular matrix between the layers of cells can be investigated using techniques well known to the skilled person, such as microscopy, e.g. light or electron microscopy. Cell death and/or cell viability can be determined by using methods well known to the skilled person, for example by analysis of caspase expression and/or activity. Transfer of molecules across the multilayer sheet of cells can be investigated as described hereinabove.

In some embodiments, one or more of the above properties may be investigated after the multilayer sheet of cells has been subjected to a flow rate of one of 300 to 800, 350 to 700, or 400 to 600 μl/min, for example one of about 540 μl/min 480 μl/min, 450 μl/min or 430 μl/min. In some embodiments, one or more of the above properties may be investigated after the multilayer sheet of cells has been subjected to a flow conditions for one of about 1-10 minutes, 10-30 minutes, 0.5-2 hours, 1-4 hours, 2-6 hours, 6-12 hours, 12-24 hours, 1-14 days, 2-12 days, 3-10 days, 4-7 days, and about 5 days.

In some embodiments, the layers of the multilayer sheet of the invention may be more distinct (i.e. there may be less intermingling of cells of different layers) when the multilayer sheet of cells is cultured under flow conditions, as compared to when the multilayer sheet of cells is cultured in the absence of flow conditions.

In some embodiments, the multilayer sheet of cells of the invention can be characterised in terms of mechanical properties.

In some embodiments, the multilayer sheet of cells is capable of being manipulated to a three-dimensional structure, e.g. using a mould. In some embodiments, the multilayer sheet of cells is flexible, and/or can be rolled or folded.

### Support Surfaces

In some embodiments of the present invention, the multilayer sheet of cells is provided on, or attached to, a support surface.

Any surface capable of providing structural support to the multilayer sheet of cells is contemplated.

In some embodiments, the multilayer sheet of cells may be attached or adhered to the support surface. In some embodiments, the multilayer sheet of cells may rest on the support surface.

In some embodiments, the support surface is provided adjacent the layer of endothelial cells. In some embodiments, the support surface is provide adjacent the layer of podocytes. In some embodiments, support surfaces may be provided adjacent the layer of endothelial cells and adjacent the layer of podocytes.

In particular embodiments, the layer of endothelial cells of the multilayer sheet of the invention may be attached to the support surface. Attachment to the support surface may be through cell adhesion molecules, e.g. one or more of integrins, selectins and cadherins.

In some embodiments, the support surface may be a surface suitable for the culture of endothelial cells or podocytes. In some embodiments, the surface may be suitable for attachment by endothelial cells or podocytes. In some embodiments, the support surface may be a surface to which endothelial cells attach.

In some embodiments, the support surface may be a support surface commonly used in in vitro cell culture. For example, the surface may be glass or a plastic, e.g. tissue culture polystyrene. In some embodiments, the surface may be glass or a plastic coated with one or more of collagen, poly-lysine, fibronectin, or laminin.

In some embodiments, the support surface may be e.g. a liquid/fluid permeable support surface. For example, the support surface may be a porous surface. In another example, the support may have a web or mesh structure, e.g. a fibrous web. In some embodiments a porous support surface may additionally comprise a web or mesh, e.g. overlaid on the porous support. Such support surfaces are particularly contemplated when the multilayer sheet of cells is provided in a device according to the invention.

In some embodiments, the support surface may be a surface from which the multilayer sheet of cells of the invention can be detached, e.g. without the need for enzymatic treatment, e.g. with trypsin. In some embodiments, the support surface may be a surface from which the multilayer sheet of cells can be detached without cleavage of proteins of the multilayer sheet of cells. In some embodiments, the support surface may be an environment responsive support surface, e.g. a pH responsive, electric field responsive, chemical responsive or temperature responsive support surface.

In some embodiments, the support surface may be a temperature responsive support surface. Temperature responsive support surfaces are described, for example, in Tang et al., Polymers (2012), 4: 1478-1498, which is hereby incorporated by reference in its entirety.

Briefly, temperature responsive support surfaces comprise one or more temperature-responsive polymers, which provide a hydrophobic surface at one temperature, and a hydrophilic surface at another temperature. When the surface is hydrophobic, cells can adhere and grow on the surface; when the surface is hydrophilic, cells can detach spontaneously (without the need for enzymatic treatment to detach the cells from the support surface). The temperature responsive polymer may be provided as a layer on top of a layer of tissue culture plastic or polystyrene. In some embodiments, the temperature responsive polymer may be grafted onto tissue culture polystyrene using electron beam irradiated polymerisation.

In some embodiments, the temperature responsive polymer may be Poly(N-isopropylacrylamide) (PIPAAm). Polymer chains of PIPAAm hydrate to form an expanded structure in water at a lower temperature below 32° C., and form a compact structure by dehydration at above 32° C. At 37° C., a support surface comprising tissue culture polystyrene and a surface layer of PIPAAm provides a hydrophobic surface, and at 20° C. the surface provides a hydrophilic surface. In some embodiments, the support surface may be a Nunc UpCell Surface (Thermo Scientific), which comprises PIPAAm. Methods for culturing cells on Nunc UpCell Surfaces, and releasing cells therefrom, are described for example in Thermo Scientific Nunc UpCell Surface: Cell Harvesting by Temperature Reduction (2008) 74061/N20230—Ver. 1.0—September 2008—YNI, which is hereby incorporated by reference in its entirety.

Such temperature responsive support surfaces are useful for preparing a multilayer sheet of cells according to the invention. Part or all of a multilayer sheet of cells according to the invention can be prepared on a temperature responsive support surface, and then separated therefrom by a change in temperature, yielding a multilayer sheet of cells which is physically separate from the support surface, and which has a structure (e.g. of the cell layers, extracellular matrix) which is substantially unchanged as compared to the structure of the multilayer sheet of cells when attached to the support surface.

Cell layers and/or multilayer sheets of cells according to the invention may be detached from a temperature responsive support surface by a temperature change alone, or detachment may include using e.g. a transfer membrane to facilitate removal of the cell layers and/or multilayer sheet from the temperature responsive support surface. The layer of cells or multilayer sheet of cells may be lifted from the temperature responsive support surface e.g. using tweezers, or may be floated-off by adding e.g. cell culture media.

The layer of cells or multilayer sheet of cells may be cut at the edges of the sheet to facilitate detachment from the support surface.

In particular embodiments, where the present invention provides a temperature responsive support surface, the layer of endothelial cells of the multilayer sheet of the invention is adjacent to the support surface. In some embodiments, endothelial cells of the layer of endothelial cells are attached to the temperature responsive support surface.

### In Vitro Cell Culture

The present invention also provides an in vitro cell culture, comprising a layer of endothelial cells and a layer of podocytes.

The in vitro culture is preferably of a multilayer sheet of cells according to the present invention as described herein.

The culture is of living cells, e.g. cells which are respiring. In some embodiments, the in vitro cell culture comprises culture media, e.g. liquid or gel culture media containing nutrients and/or growth factors, preferably in contact with the sheet of cells. The in vitro cell culture according to the invention may be performed in a bioreactor provided with an appropriate supply of nutrients, air/oxygen and/or growth factors, and may be maintained at a suitable temperature and/or pH. The skilled person is readily able to determine suitable conditions for an in vitro culture of a layer of endothelial cells and a layer of podocytes according to the present invention.

In some embodiments, the in vitro culture of cells may be provided with flow conditions as described herein. In some embodiments, the layers of cells of the in vitro culture may be more distinct (i.e. there may be less intermingling of cells of different layers) when the in vitro culture of cells is cultured under flow conditions, as compared to when the in vitro culture of cells is cultured in the absence of flow conditions.

In some embodiments, the in vitro culture of cells may be provided on a support surface as described herein. In some embodiments, the in vitro culture of cells may be provided in a device according to the present invention.

### Devices

In another aspect, the present invention provides a device comprising a multilayer sheet of cells according to the present invention. The device may have two compartments, separated by a multilayer sheet according to the invention.

In some embodiments, the compartments of the device will be formed by a multilayer sheet of cells of the invention separating or partitioning a chamber into a compartment which is adjacent to the layer of endothelial cells, and a compartment which is adjacent to the layer of podocytes.

The sheet may be in contact with liquid/fluid in each compartment, and may prevent liquid/fluid flowing between the compartments, but allow for substances capable of being filtered to pass between the compartments. The sheet effectively forms a semi-permeable membrane separating the two compartments; i.e. the sheet prevents liquid/fluid communication between the two compartments.

In some embodiments, the compartments may be substantially completely separated by the multilayer sheet of cells. In some embodiments, the compartments may prevent passage of materials from one compartment to another compartment of the device except through the multilayer sheet of cells.

In some embodiments, the device may not allow passage of large proteins from one compartment to another compartment. In some embodiments, the device may not allow passage of proteins having a size larger than about one of 70 kDa, 80 kDa, 90 kDa or 100 kDa from one compartment to another compartment. In some embodiments, the device may allow passage of one or more of small molecules, water and ions between compartments. For example, in some embodiments, the device may allow passage of one or more of urea, creatinine, or cystatin C.

In some embodiments, the device (a) may not allow passage of proteins having a size larger than about one of 70 kDa, 80 kDa, 90 kDa or 100 kDa from one compartment to another compartment, and (b) may allow passage of one or more of small molecules, water and ions, e.g. urea, creatinine, and/or cystatin C, from one compartment to another.

Whether a device allows passage of molecules between compartments can be investigated as described herein above for the multilayer sheet of cells.

In some embodiments, the compartments of the device according to the invention may be disposed one above the other, the multilayer sheet of cells separating an upper compartment and a lower compartment. In some embodiments, the upper compartment is adjacent the layer of endothelial cells and the lower compartment is adjacent the layer of podocytes. In some embodiments, the upper compartment is adjacent the layer of podocytes and the lower compartment is adjacent the layer of endothelial cells.

In some embodiments, the compartments of the device according to the invention may be disposed side-by-side, the multilayer sheet of cells separating adjacent compartments.

In some embodiments, the multilayer sheet of cells may be formed to a tube, in which the inner surface of the tube is formed one of the cell layers, and the outer surface of the tube is formed another cell layer. In such embodiments, the lumen of the tube and the external of the tube are the two compartments. In some embodiments, the inner cell layer of the tube may be the layer of endothelial cells, and the outer cell layer of the tube may be the layer of podocytes. In some embodiments, the inner cell layer of the tube may be the layer of podocytes, and the outer cell layer of the tube may be the layer of endothelial cells.

In some embodiments, the multilayer sheet of cells may be provided as a membrane spanning an aperture. In some embodiments, the membrane may be attached at to the body defining the aperture. In some embodiments, the multilayer sheet of cells may be substantially unsupported in the region of the multilayer sheet of cells spanning the aperture.

In some embodiments, the device additionally comprises a support for the multilayer sheet of cells. In some embodiments, the support may be any support capable of providing structural support to the multilayer sheet of cells. In some embodiments, the multilayer sheet of cells may rest on the support in the device of the invention. In some embodiments, the multilayer sheet of cells may be adhered or attached to the support.

In some embodiments, the support is provided adjacent the layer of endothelial cells. In some embodiments, the support is provide adjacent the layer of podocytes. In some embodiments, supports may be provided adjacent the layer of endothelial cells and adjacent the layer of podocytes.

In some embodiments, the support may be e.g. a liquid/fluid permeable support. For example, the support may be a porous support. In some embodiments, the support allows passage of large proteins (e.g. having a size larger than about one of 70 kDa, 80 kDa, 90 kDa or 100 kDa) from one side of the support to the other side of the support (e.g. from the compartment to the multilayer sheet of cells).

In some embodiments, the compartments may be provided with a transwell-type arrangement. Transwells are known to the skilled person, and are described for example in Transwell Permeable Supports Selection and Use Guide, Corning Incorporated (2013) CLS-CC-007W, Rev6, which is hereby incorporated by reference in its entirety. For example, the multilayer sheet of cells according to the invention may be provided on a porous support of an upper compartment of a transwell.

In some embodiments, the device according to the present invention may provide for applying liquid/fluid to the multilayer sheet of cells. In some embodiments, the device may comprise a liquid/fluid inlet to one or more of the compartments of the device, and one or more liquid/fluid outlets from one or more of the compartments of the device. In some embodiments, the device comprises a liquid/fluid inlet to the compartment adjacent the layer of endothelial cells, and a liquid/fluid outlet from the compartment adjacent the layer of podocytes. In some embodiments, the device comprises a liquid/fluid inlet to the compartment adjacent the layer of endothelial cells, a liquid/fluid outlet from the compartment adjacent the layer of endothelial cells, and a liquid/fluid outlet from the compartment adjacent the layer of podocytes.

In some embodiments, the device may comprise means for applying a liquid/fluid to the device. For example, the device may comprise a pump for introducing a liquid/fluid to a device according to the invention, e.g. through a liquid/fluid inlet. In some embodiments, the device may comprise means for applying liquid/fluid to the device, and means for removing liquid/fluid from the device.

In particular embodiments, the device may be suitable for filtering a liquid/fluid for certain molecules. For example, where the device does not allow passage of large proteins (e.g. having a size larger than about one of 70 kDa, 80 kDa, 90 kDa or 100 kDa), and/or allows passage of one or more of small molecules, water and ions, e.g. urea, creatinine, and/or cystatin C from one compartment to another, the device may be suitable for separating (e.g. filtering) large proteins from small molecules and ions in a liquid.

In some embodiments, the device may comprise means for applying liquid/fluid to the device such as to allow the multilayer sheet of cells to filter the liquid/fluid provided to a filtrate comprising different amounts of one or more of proteins, large proteins, small molecules and ions as compared to the liquid/fluid applied to the device. In some embodiments, the device may comprise means for removing filtrate from the device.

In some embodiments the device comprises means for keeping the filtrate and the filtrated liquid/fluid physically separate.

In some embodiments, the device of the present invention may comprise renal epithelial cells as described herein above.

In some embodiments, the renal epithelial cells are provided in a compartment. In some embodiments, the renal epithelial cells are provided as a layer of cells.

In some embodiments, the compartment containing the renal epithelial cells may be separate to the compartments formed by the multilayer sheet of cells. In some embodiments, the compartment containing the renal epithelial cells may be in liquid/fluid communication with the compartment adjacent the layer of podocytes.

In some embodiments, the device may provide for applying filtrate from filtration by a multilayer sheet of cells according to the present invention to renal epithelial cells.

In some embodiments, the renal epithelial cells may be provided as a layer of cells dividing the compartment containing the renal epithelial cells into two subcompartments. In some embodiments, the subcompartments may be substantially completely separated by the layer of renal epithelial cells. In some embodiments, the subcompartments may prevent passage of materials from one subcompartment to another subcompartment of except through the layer of renal epithelial cells.

In some embodiments, the subcompartments may be disposed one above the other, the layer of renal epithelial cells separating an upper subcompartment and a lower subcompartment.

In some embodiments, the device additionally comprises a support for the layer of renal epithelial cells. In some embodiments, the support may be any support capable of providing structural support to the layer of renal epithelial cells. In some embodiments, the layer of renal epithelial cells may rest on the support. In some embodiments, the layer of renal epithelial cells may be adhered or attached to the support. A suitable support may be a porous support or a web, mesh. Preferably the support allows for the passage or transfer of fluid from/to the cells.

In some embodiments, the subcompartments may be provided with a transwell-type arrangement, having the layer of renal epithelial cells provided on a porous support of an upper subcompartment.

In some embodiments, a device of the present invention may be, or may be comprised in, an artificial kidney.

An artificial kidney as used herein refers to a non-naturally occurring product capable of dialysing fluid, e.g. blood or plasma.

In some embodiments, an artificial kidney may be a dialysis apparatus, e.g. a haemodialyis apparatus. That is, in some embodiments, the multilayer sheet of cells of the present invention may be comprised in (e.g. may be a component part of) a dialysis machine.

In some embodiments, an artificial kidney may comprise bioengineered kidney tissue.

In some embodiments, an artificial kidney may be an implantable or wearable dialysis apparatus.

Kidney function and dialysis are well known to the skilled person, and are described, for example, in Handbook of Dialysis. 4th ed. New York, N.Y.; 2008, which is hereby incorporated by reference in its entirety.

An artificial kidney typically comprises a fluid inlet to the artificial kidney for blood to be dialysed, a dialyser part wherein blood is dialysed, and a fluid outlet part for dialysed blood to leave the artificial kidney. The dialyser part may comprise, or may consist of, a removable cartridge providing the blood or plasma filtering part of the artificial kidney. The cartridge may be consumable.

In the artificial kidney of the invention, the multilayer sheet of cells of the present invention may provide for the blood or plasma filtering function of the artificial kidney. That is, the artificial kidney may comprise a dialyser part comprising a multilayer sheet of cells of the present invention. The part may comprise, or may consist of, a removable cartridge, and may be consumed by the filtering/dialysing process.

In addition to the multilayer sheet of cells or device according to the present invention, the artificial kidney may comprise any other components necessary for the artificial kidney to function to dialyse blood or plasma.

In some embodiments, devices of the present invention may be microfluidic devices.

### Uses of the Multilayer and Devices

In another aspect, the present invention provides a multilayer sheets of cells or device of the present invention for use in an assay, e.g. an in vitro assay.

The assay may be an assay relevant to kidney tissue or function. Assays include, for example, toxicity assays (e.g. drug toxicity assays), kidney tissue damage assays, kidney function assays, etc.

In some embodiments, the multilayer sheets of cells and devices of the present invention may be useful to study the structure and function of the renal corpuscle filtration barrier. For example, the multilayer sheet of cells or components thereof (e.g. cells, layers of cells, extracellular matrix) may be manipulated to have modified expression of one or more molecules expressed by cells of the multilayer sheet of cells. For example, expression of a molecule which is expressed by a cell of the multilayer sheet of cells of the invention may be downregulated or upregulated, and the effect of downregulation/upregulation on the structure and/or function of the multilayer sheet of cells or component thereof can be investigated.

In some embodiments, the multilayer sheets of cells and devices of the present invention may be useful to study renal disease, e.g. diseases effecting the renal corpuscle filtration barrier, and/or conditions associated with damage or dysfunction of the renal corpuscle filtration barrier, e.g. proteinuria. For example, the multilayer sheet of cells or components thereof may be manipulated to have modified expression of one or more molecules expressed by cells of the multilayer sheet of cells, the modified expression reflecting modified expression occurring in a particular disease or disease state. The effect of the disease or disease state associated expression of the molecule on structure and/or function of the multilayer sheet of cells or component thereof can then be reflecting modified expression occurring in a particular disease or disease state investigated.

For example, the multilayer sheets of cells and devices may be useful to study renal disease.

For example, the multilayer sheet of cells or components thereof may be manipulated to have modified expression of one or more proteins expressed by the cells. For example, expression of a protein which is expressed by a cell of the multilayer sheet of cells of the invention may be downregulated or upregulated.

In some embodiments, the protein which is expressed by a cell of the multilayer sheet of cells may be a marker for that cell type. In some embodiments, the protein may be a protein whose expression is specific to that cell type. Multilayer sheets of cells comprising cells having modified expression of a protein which is ordinarily expressed by a cell of the multilayer sheet of cells of the invention may be useful to investigate the effect of modified expression of the protein on the cell or multilayer sheet of cells, and may therefore by useful to investigate function of the marker.

An in vitro assay according to the present invention may involve analysing the effect of a particular treatment or condition on a property of the multilayer sheet of cells of the invention, or component thereof. A component of a multilayer sheet of cells of the invention may be an individual cell, plurality of cells, layer of cells, extracellular matrix, etc.

Also provided are methods for performing an in vitro assay, comprising applying a particular treatment or condition to a multilayer sheet of cells or device of the present invention, and analysing the effect of the treatment or condition on a property of the multilayer sheet of cells of the invention, or component thereof.

For example, the assay may involve investigating the effect of a treatment or condition on one or more of: the morphology of cells of the multilayer sheet of cells, gene and/or protein expression by cells of the multilayer sheet of cells, surface marker expression by cells of the multilayer sheet of cells, proteins secreted by cells of the multilayer sheet of cells, viability of cells of the multilayer sheet of cells, cell death of cells of the multilayer sheet of cells, percent confluence of the layers of cells of the multilayer sheet of cells, junctions between cells in cell layers, composition and/or thickness of the extracellular matrix disposed between the layers of cells of the multilayer sheet of cells, tensile strength of the multilayer sheet of cells, and flow tolerance of the multilayer sheet of cells.

These properties can be investigated by methods described herein, or methods which are otherwise well known to the skilled person.

Assays may involve analysis of one or more of the physical structure of a multilayer sheet of cells or component thereof, functional properties of a multilayer sheet of cells or component thereof, liquid/fluid adjacent the layer of endothelial cells, liquid/fluid adjacent the layer of podocytes, liquid/fluid flowed over the multilayer sheet of cells, and liquid/fluid filtered through the multilayer sheet of cells. The liquid/fluid analysed in the assays may be e.g. one or more of cell blood, plasma, blood or plasma filtrate, of cell culture media.

In some embodiments, the methods may comprise detection of biomarkers of stress or damage to the multilayer sheet of cells or constituents thereof.

An in vitro assay according to the invention may involve analysing the effect of a particular treatment or condition on the function of the multilayer sheet of the invention, or component thereof (e.g. a cell, cell layer, extracellular matrix, etc.).

For example, the assay may involve investigating the effect of a treatment or condition on permeability of the multilayer sheet of cells to one or more molecules (e.g. filtration function). Whether a multilayer sheet of cells (or cell layer or extracellular matrix thereof) allows passage of a given molecule from one side to another can be investigated for example as described herein. In some embodiments, the assay measures transfer of a substance across the multilayer sheet of cells.

In some embodiments, the treatment may be treatment with a drug, and the assay may be a drug toxicity assay.

In some embodiments, the multilayer sheet of cells or devices of the invention may be used in a nephrotoxicity assay. Nephrotoxicity assays are well known to the skilled person, and are described for example in Huang et al., Expert Opin Drug Metab Toxicol (2014), 10(12): 1621:1635, which is hereby incorporated by reference in its entirety.

In some embodiments, an in vitro assay according to the invention may be an assay capable of detecting a change in liquid/fluid adjacent the multilayer sheet of cells of the invention, or a cell layer thereof. In some embodiments, the in vitro assay may comprise qualitative and/or qualitative determination of a molecule in a liquid/fluid adjacent the layer of endothelial cells, and/or a liquid/fluid adjacent the layer of podocytes. Qualitative/quantitative determinations of a molecule in a liquid/fluid can be made by any suitable means, such as by immunoassay, mass spectrometry, etc.

The present invention also provides the use of a multilayer sheet of cells, device, artificial kidney or dialysis apparatus according to the invention to filter blood or plasma.

In some embodiments, use to filter blood or plasma is in an assay, e.g. an in vitro or ex vivo assay. In some embodiments, the blood or plasma may be a blood or plasma sample which has been obtained from an individual. In some embodiments, the blood or plasma sample may be a donor sample. In some embodiments, the plasma sample may be a patient sample.

In a related aspect, the present invention also provides a method for filtering a fluid/liquid, e.g. a bodily fluid such as blood or plasma, the method comprising applying blood or plasma to a multilayer sheet of cells, device, artificial kidney or dialysis apparatus according to the invention, and using multilayer sheet of cells to filter the blood or plasma.

In some embodiments, the method comprises applying the blood or plasma to the surface of the multilayer sheet of cells, e.g. to the layer of endothelial cells, and allowing filtrate to form at the side of the multilayer sheet of cells adjacent the layer of podocytes.

In some embodiments, the method comprises applying the blood or plasma to the surface of the multilayer sheet of cells under pressure, e.g. to the layer of endothelial cells. In some embodiments the method comprises flowing the blood or plasma over the surface of the multilayer sheet of cells. In some embodiments, the method comprises flowing the blood or plasma over the layer of endothelial cells.

In some embodiments, the method comprises collecting filtered blood or plasma. In some embodiments the method comprises collecting filtered blood or plasma from the side of the multilayer sheet of cells adjacent the layer of endothelial cells. In some embodiments, the method comprises collecting filtrate from the side of the multilayer sheet of cells adjacent the layer of podocytes.

### Methods of Treatment and Medical Uses

In another aspect, the present invention provides a method of medical treatment using a multilayer sheet of cells, device, artificial kidney or dialysis apparatus of the invention. Also provided is the multilayer sheet of cells, device, artificial kidney or dialysis apparatus of the invention for use in a method of treatment. The treatment is preferably dialysis, e.g. haemodialysis.

The subject is a patient in need of dialysis. The patient is preferably a mammal, e.g. a human.

A subject for treatment according to the present invention may have impaired kidney function. For example, the patient may be suffering from kidney failure. A patient may have impaired kidney function as a result of chronic kidney disease (CKD), or acute kidney injury (AKI).

Chronic kidney disease may be caused by one or more of hypertension, vascular disease (e.g. bilateral renal artery steonisis, ischemic nephrothapy, hemolytic-uremic syndrome, vasculitis), glomerular disease (e.g. focal glomerulosclerosis, glomerulonephritis, diabetic nephrothapy, lupus nephritis), polycystic kidney disease, tubulointerstitial disease (e.g. tubulointerstitial nephritis, reflux nephropathy), obstructive nephrothapy, infection, or may have unknown cause.

Acute kidney injury may be caused by one or more of systemic disease (e.g. autoimmune disease), physical injury to kidney tissue, percutaneous coronary intervention (PCI) using a contrast agent, dehydration, infection (sepsis), and antibiotic treatment.

In the methods, the multilayer sheet of cells, device, artificial kidney or dialysis apparatus of the invention is used to dialyse blood or plasma. In some embodiments, the dialysed blood or plasma is returned to the same body.

In some embodiments, a patient is connected to a machine to flow blood or plasma to be dialysed to a multilayer sheet of cells, device, artificial kidney or dialysis apparatus of the invention, and to return dialysed blood or plasma to the patient's body.

In some embodiments, an individual multilayer sheet of cells, or multilayer sheet of cells of a device, artificial kidney or dialysis apparatus of the invention is provided for use by a single patient. In some embodiments, an individual multilayer sheet of cells, or multilayer sheet of cells of a device, artificial kidney or dialysis apparatus of the invention is provided for a single use.

In some embodiments, an individual multilayer sheet of cells, or multilayer sheet of cells of a device, artificial kidney or dialysis apparatus of the invention is consumed (i.e. used-up) by the filtration/dialysis process.

The present invention also provides a transplant tissue or tissue graft comprising a multilayer sheet of cells according to the present invention. The present invention also provides a multilayer sheet of cells according to the present invention for use as a tissue for a transplant or as a tissue graft.

### Methods for Producing Sheets

The present invention also provides methods for producing a multilayer sheet of cells according to the present invention.

The methods comprise: (i) providing a layer of endothelial cells; (ii) providing a layer of podocytes; and (iii) culturing the cells in vitro; thereby producing a multilayer sheet of cells.

In some embodiments, providing a layer of endothelial cells comprises providing endothelial cells to a support surface, and culturing the cells in vitro.

In some embodiments, providing a layer of endothelial cells comprises contacting endothelial cells with a support surface, and culturing the cells on the support surface in vitro. In some embodiments, the endothelial cells may be attached or adhered to the support surface. In some embodiments, the endothelial cells may rest on the support surface.

In some embodiments, the support surface is a surface from which the multilayer sheet of cells of the invention can be detached, e.g. without the need for enzymatic treatment, e.g. with trypsin. In some embodiments, the support surface may be a surface from which the multilayer sheet of cells can be detached without cleavage of proteins of the multilayer sheet of cells.

In some embodiments, the support surface may e.g. be a pH responsive, electric field responsive, chemical responsive or temperature responsive support surface. In some embodiments, the support surface may be a temperature responsive support surface, e.g. a support surface comprising PIPAAm. In some embodiments, the support surface may be a Nunc UpCell Surface (Thermo Scientific).

In some embodiments, providing a layer podocytes comprises providing podocytes to a layer of endothelial cells, and culturing the cells in vitro. In some embodiments, the layer of endothelial cells to which the podocytes are provided is a confluent layer of endothelial cells. In some embodiments, the methods comprise culturing the podocytes for sufficient time and under suitable conditions to form a confluent layer of cells on top of the layer of endothelial cells. The skilled person is able to determine suitable conditions for the culture of endothelial cells and podocytes by reference to, for example, Byron et al. J Am Soc Nephrol (2014) 25: 953-966, which is hereby incorporated by reference in its entirety.

In some embodiments, the multilayer sheet of cells comprises an extracellular matrix disposed between the layer of endothelial cells and the layer of podocytes. In some embodiments, the methods comprise culturing the endothelial cells and podocytes to allow deposition of an extracellular matrix between the layer of endothelial cells and the layer of podocytes.

In some embodiments, the methods additionally comprise a step of (iv) detaching the multilayer sheet of cells from the support surface. In some embodiments, detachment does not involve enzymatic treatment. In some embodiments, detachment may involve applying a condition to the multilayer sheet of cells provided on a condition responsive support surface. In some embodiments, the methods comprise changing the temperature of the multilayer sheet of cells and/or support effective to cause or facilitate detachment of the multilayer sheet of cells from the support surface.

In embodiments where the support surface is a temperature responsive support surface, e.g. a support surface comprising PIPAAm, the method may comprise lowering the temperature of the multilayer sheet of cells or a layer of cells and/or support surface to around 20° C.

Cell layers and/or multilayer sheets of cells according to the invention may be detached from a temperature responsive support surface by a temperature change alone, or detachment may include using e.g. a transfer membrane to facilitate removal of the cell layers and/or multilayer sheet from the temperature responsive support surface. In some embodiments, detaching a cell layer and/or multilayer sheet cells from a temperature responsive support surface may comprise lifting the layer or sheet from the temperature responsive support surface e.g. using tweezers. In some embodiments, detaching a cell layer and/or multilayer sheet cells from a temperature responsive support surface may comprise floating the layer or sheet off the temperature responsive support surface by adding e.g. cell culture media to the cell culture. In some embodiments, detaching the cells may comprise a step of cutting the layer of cells or multilayer sheet of cells at the edges of the sheet, to facilitate detachment from the support surface.

In some embodiments, the methods additionally comprise forming the detached multilayer sheet of cells of the invention to a three-dimensional structure. Forming the multilayer sheet of cells to a three-dimensional structure may comprise, for example, contacting the multilayer sheet of cells with a mould, to adopt the shape of the mould. Forming the multilayer sheet of cells to a three-dimensional structure may comprise rolling or folding the multilayer sheet of cells to a desired shape.

The multilayer sheet of cells may be formed e.g. to a structure resembling the structure of the naturally-occurring renal corpuscle filtration barrier. In some embodiments, the multilayer sheet of cells may be formed to a structure appropriate to its intended use, e.g. in a device, artificial kidney or dialysis apparatus of the invention, in an in vitro assay, for dialysis of blood or plasma, for treatment by dialysis etc. In some embodiments, the methods comprise forming the multilayer sheet of cells to a structure which mimics the tubular filtration regions of the kidney, by bending the sheet around a temporary mould that can be removed once the structure is formed.

In some embodiments, the methods may additionally comprise preparing the multilayer sheet of cells for storage or delivery. In some embodiments, the methods may include freezing the multilayer sheet of cells, and may optionally further comprise storing and/or transporting the frozen cells until such time as they are required to be used.

The present invention also provides a multilayer sheet of cells obtained or obtainable by a method according to the present invention.

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

### BRIEF DESCRIPTION OF THE FIGURES

Embodiments and experiments illustrating the principles of the invention will now be discussed with reference to the accompanying figures:

FIG. 1. Photographs showing a sheet of GEnCs (passage 27) on a confluent layer of podocytes (AB passage 20). “Anti-PECAM” panels show staining of cells with an anti-PECAM antibody. “Anti-synaptapodin” shows staining of cells with an anti-synaptapodin antibody. “Actin” shows staining of cells for actin. “Merge” top right panel shows the merge of the top left and top middle images. “Merge” bottom right panel shows the merge of the bottom left and bottom middle images.

FIG. 2. Electron micrographs showing a sheet of glomerular endothelial cells (GEnCs; (passage 31) on a layer of podocytes (GFP-AB passage 15+14). “GEnC” indicates a glomerular endothelial cell. “Aclar” indicates aclar.

FIG. 3. Photographs showing podocytes (AB passage 25) cultured for 24 h under static (top panels) or flow conditions (lower panels) at a flow rate of 500 μl/min. “Vinculin” shows staining of cells for vinculin, “Actin” shows staining of cells for actin, and “Merge” shows the merge of vinculin and actin staining images.

FIG. 4. Photographs showing podocytes (GFP-AB passage 15+10) and GEnC (passage 30) under static conditions (upper two rows of panels) and flow conditions (lower two rows of panels). “Dapi” indicates staining of the cells with Dapi. “GFP” indicates GFP fluorescence signal from the cells. “PECAM” panels show staining of cells with an anti-PECAM antibody. “Merge” panels show merges of Dapi, GFP and PECAM images for each row of panels.

FIG. 5. Electron micrographs of podocytes (nephrin-FLAG; passage 18+13) cultured under static conditions (left column panels) and flow conditions (right column panels). Arrows indicate concentrations of actin fibres.

FIG. 6. Schematic of an exemplary embodiment of a flow chamber (101), divided into two compartments (102, 103) by a bilayer cell sheet (104). The upper compartment (102) has an inlet (105) and an outlet (106) allowing fluid to flow over endothelial cells of the sheet. The lower compartment provides for collection of filtrate and has an outlet (107) allowing filtrate to be removed.

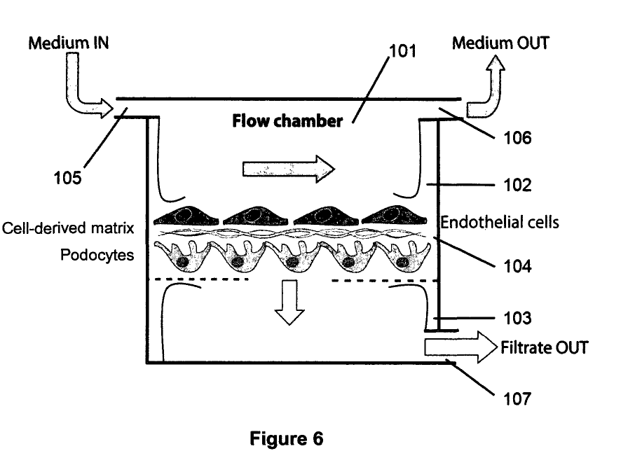

FIGS. 7A and 7B. Diagram illustrating generation of self-standing cell sheets generated by culturing cells to confluence on temperature sensitive surfaces. (A) Culture on substrate having poly(N-isopropylacrylamid)(NIPAAm) chains grafted to the substrate surface at 37° C. provides for cell attachment. (B) NIPAAm chains become hydrophilic at 21° C. causing them to swell, de-bond the cells and enable release of a cell sheet.

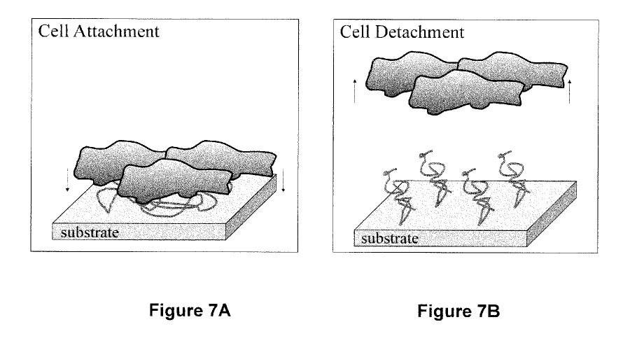

### EXAMPLES

The following examples demonstrate that:

(1) Glomerular endothelial cells (GEnCs) can be grown on thermo-responsive surfaces and lifted off as cell sheets (see Example 1.2)
(2) Podocytes and proximal tubule cells do not grow well on thermo-responsive surfaces (see Examples 1.1 and 1.3)
(3) Podocytes can be grown on a layer of GEnCs (see Examples 1.4 and 2.3)
(4)(i) GEnCs can be cultured to confluence on a temperature responsive surface, (ii) a layer of podocytes can be cultured on top of the GEnC layer to form a bilayer of GEnCs and podocytes, and (iii) the bilayer can be detached from the temperature responsive surface as a bilayer sheet of cells (see Example 1.4).
(5) Podocytes and GEnC have different morphological appearances and distribution of focal adhesion/junctional proteins under flow conditions as compared to the cells grown under static conditions (see Examples 3-5).
(6) Podocytes and glomerular endothelial cells are viable in coculture, under both static and flow conditions (see Examples 4 and 5)

### Example 1: Cell Sheets from Temperature-Responsive Dishes—Analysis by Light Microscopy

Cell sheets were prepared on temperature-responsive UpCell Surface cell culture dishes (Thermo Scientific Nunc), which comprises PIPAAm, according to the manufacturer's instructions.

Briefly, cells were seeded in cell culture media on UpCell Surface cell culture dishes and cultured in vitro to confluence at 37° C. For harvesting the cell layer, the cell culture media was aspirated, and about 0.5 ml of cooled or room temperature cell culture media was added to the cells to reduce the temperature of the cells and culture dishes.

A transfer membrane was applied to the surface of the cultured cells, and the cultured cells and culture dishes were allowed to cool to provide the temperature responsive dish with a hydrophilic surface.

Cell sheets were then removed from the UpCell Surface cell culture dishes by lifting up the transfer membrane, with the layer of cultured cells attached to it.

Where cell sheets were transferred to another layer of cells in cell culture, the sheet of cells attached to the transfer membrane was placed onto a layer of cells in culture. The sheet of cells was allowed to attach to the cells in culture, and additional cell culture media was added to facilitate detachment of the transfer membrane from the bilayer of cells. The transfer membrane was then removed from the culture.

### 1.1 Podocytes do not Form a Detachable Cell Sheet on Temperature-Responsive (TR) Dishes

Podocytes do not grow well on polymer on temperature responsive dishes. Cells were very patchy and unhealthy looking after seeding on dishes. Coating the dishes with the glomerular extracellular matrix (ECM) ligand collagen IV did not improve growth of podocytes on temperature responsive dishes.

### 1.2 Glomerular Endothelial Cells (GEnCs) Grow Well on TR Dishes.

Glomerular endothelial cells grow well on temperature responsive dishes, and formed a detachable cell sheet after one week at 33° C. (split one from a confluent T75 flask of cells, onto two 3.5 cm dishes).

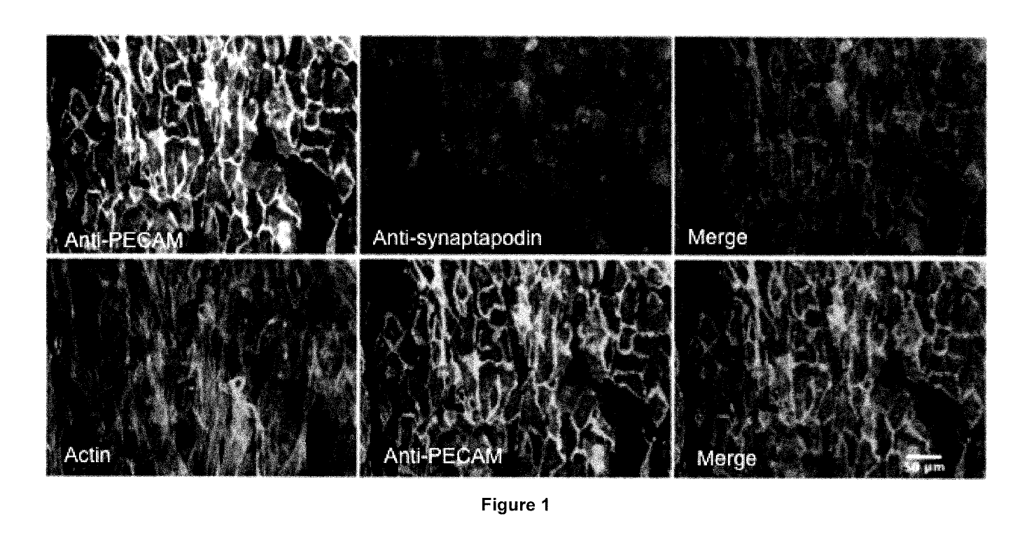

Cell sheets were cut and transferred onto confluent layer of podocytes grown on coverslips and aclar. Cells were incubated at 37° C. for 14 days. Subsequently, cocultured cells were stained with synaptopodin (a podocyte marker) and with PECAM1 (a GEnC marker). Both cell types could be detected (FIG. 1).

### 1.3 Proximal Tubular Epithelial Cells do not Grow Well on TR Dishes

HK-2 (proximal tubular epithelial cells) do not grow very well on temperature responsive dishes. Cells were very round and most of the cells did not attach to the dish. The manufacturer's recommendation is not to grow these cells to confluence.

### 1.4 Preparing a Bilayer of a GEnC Sheet of Cells and a Layer of Podocytes on a TR Dish

GEnCs were cultured in vitro to a confluent monolayer of cells on a temperature responsive dish. Podocytes were subsequently added to the cell culture, and cultured in vitro to provide a confluent monolayer of cells on top of the layer of GEnCs.

Subsequently, the cells and culture dishes were cooled to provide the temperature responsive dish with a hydrophilic surface for detachment of the bilayer of GEnCs and podocytes.

Remarkably, the GEnC+podocyte bilayer detached from the temperature responsive dish as a single sheet of the bilayer of cells. The bilayer could be detached from the temperature responsive surfaces using tweezers, and could also be floated-off the surfaces with cell culture media.

### Example 2: Analysis of Cell Sheets by Electron Microscopy

For improved imaging of cultures of GEnC and podocytes, cocultures were imaged grown on aclar and prepared for electron microscopy (EM). Transmission EM was performed using a Tecnai 12 Biotwin.

### 2.1 GEnC Sheets (Passage 29) Placed on a Layer of Podocytes (GFP-AB Passage 15+10)

GEnC (passage 29) sheets were placed on a layer of podocytes (GFP-AB passage 15+10), and the cells were differentiated for 14 days. Overall the cells were not healthy, there was evidence of significant cell death, and the podocytes appeared flatter than normal.

### 2.2 GEnC Sheets (Passage 31) Placed on a Layer of Podocytes (GFP-AB Passage 15+10)

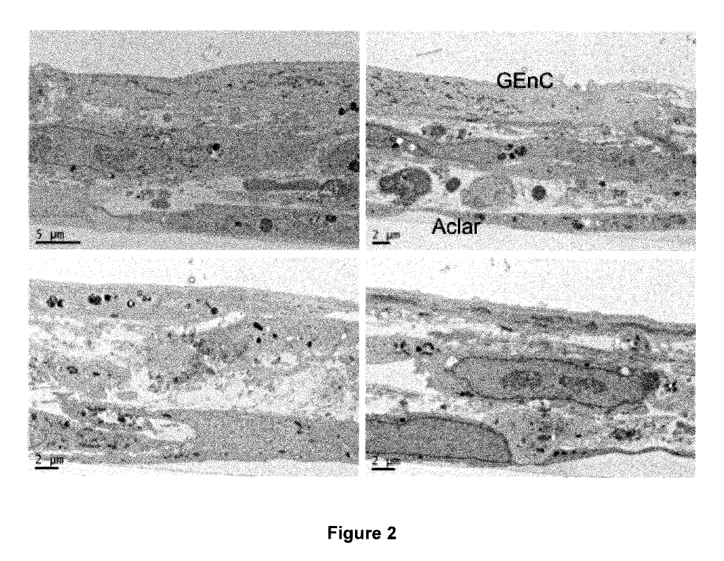

GEnC (passage 31) sheets were placed on a layer of podocytes (GFP-AB passage 15+14). Cells were left to differentiate for 14 days. There was lots of cell death and podocytes appeared flatter than usual (FIG. 2).

2.3 GEnC Sheets (Passage 31) Differentiated for 7 Days, and Layered with Podocytes (GFP-AB Passage 15+10)

GEnC (passage 27) were differentiated for 7 days, and then podocytes (GFP-AB passage 15+10) were seeded onto the GEnC layer. The cells were then differentiated for another 7 days. Cells were healthy and separated.

In view of this result, and the successful production of a bilayer as described in 1.4 above, it was therefore decided that for preparing bilayers of cells, the method would be to culture a layer of glomerular endothelial cells, and to then layer and culture podocytes on top of the layer of glomerular endothelial cells, to produce a bilayer of glomerular endothelial cells and podocytes.

### Example 3: Analysis of Podocytes Under Flow Conditions by Light Microscopy

QuasiVivo flow chambers were used to investigate whether podocytes can tolerate flow conditions. Using the QuasiVivo chambers cells were cultured under static and flow conditions. A variable flow rate was used over a time course. Imaging was performed with a slide scanner.

3.1 Podocytes (AB Passage 24) Cultured for 24 Hours at a Flow Rate of 480 μl/Min

Podocytes (AB passage 24) were cultured under flow conditions at a flow rate of 480 μl/min, or under static conditions, for 24 hours. Cells cultured under flow conditions formed a more complete monolayer (i.e. were more confluent) than cells cultured under static conditions seeded at the same density.

3.2 Podocytes (AB Passage 25) Cultured for 24 Hours at a Flow Rate of 500 μl/Min

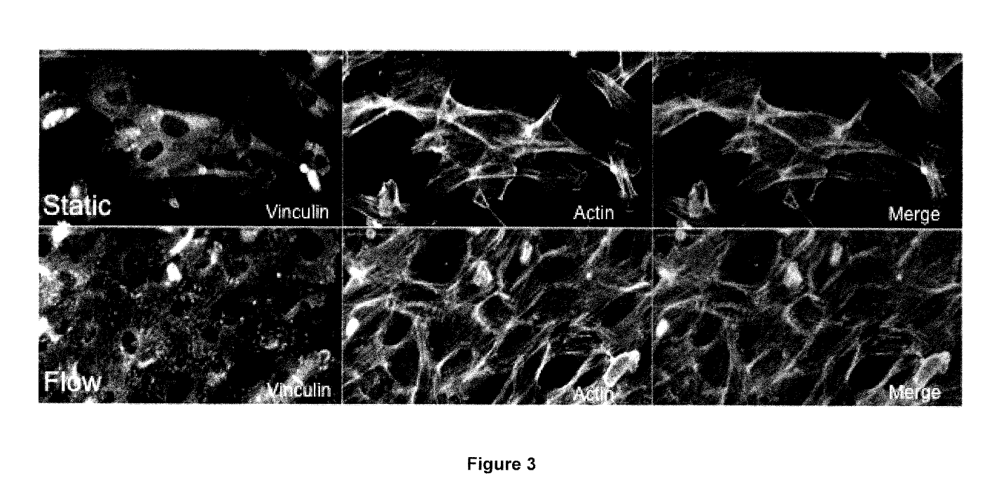

Podocytes (AB passage 25) were cultured under flow conditions at a flow rate of 500 μl/min, or under static conditions, for 24 hours. Cells cultured under flow conditions were more confluent than cells cultured under static conditions seeded at the same density (FIG. 3).

3.3 Podocytes (AB Passage 26) Cultured for 7 Days at a Flow Rate of 450 μl/Min

Podocytes (AB passage 26) were cultured under flow conditions at a flow rate of 450 μl/min, or under static conditions, for 7 days. Cells cultured under flow conditions were more confluent than cells cultured under static conditions seeded at the same density, but the difference was less pronounced than at 24 hours. Focal adhesions (FAs) of the podocytes were longer and more abundant for cells cultured under flow conditions, and the localisation of vinculin was primarily to the FAs.

3.4 Podocytes (Nephrin-FLAG, Passage 18+11) Cultured for 5 Days at a Flow Rate of 430 μl/min

Podocytes having stable expression FLAG-tagged nephrin (passage 18+11) were cultured for 5 days at a flow rate of 430 μl/min, or under static conditions. Overall nephrin expression was increased in cells cultured under flow conditions relative to cells cultured under static conditions.

3.5 Podocytes (Nephrin-FLAG, Passage 18+13) Cultured for 5 Days at a Flow Rate of 450 μl/min

Podocytes having stable expression FLAG-tagged nephrin (passage 18+13) were cultured for 5 days at a flow rate of 450 μl/min, or under static conditions and analysed for surface nephrin expression (cells were not permeabilised prior to imaging).

### Example 4: Analysis of GEnCs and Podocytes Under Flow Conditions by Light Microscopy

QuasiVivo flow chambers were used to investigate whether GEnCs and podocytes can tolerate flow conditions. Using the QuasiVivo chambers cells were cultured under static and flow conditions. A variable flow rate was used over a time course. Imaging was performed with a slide scanner.

### 4.1 Coculture of Glomerular Endothelial Cells (Passage 31) and Podocytes (Nephrin-FLAG, Passage 18+13)

Glomerular endothelial cells (passage 31) were grown for 8 days at 37° C. on cover slips. Podocytes (nephrin Flag passage 18+13) were added, and cells were then cultured under flow conditions or static conditions for 5 days. Nephrin expression was increased in cells cultured under flow conditions relative to cells cultured under static conditions.

### 4.2 Coculture of Glomerular Endothelial Cells (Passage 30) and Podocytes (GFP-AB, Passage 15+10)

Three QuasiVivo flow chambers were used in this flow experiment. One chamber for podocytes, one chamber for endothelial cells and one chamber for both cell types together. The flow rate was 540 μl/min and the same number of cells were used for the static control. Cells were incubated for 5 days at 37° C. under static or flow conditions.

### Podocytes

Under static conditions the actin filaments of the podocytes were very long and followed a parallel direction. Under flow conditions, the actin filaments were a lot shorter and looked more like a star shape with a centre from which the filaments spread out.

### Endothelial Cells

Actin filaments displayed a radial arrangement under flow conditions, but this was not as pronounced as in the podocytes. Under static conditions, PECAM1 appeared long and thin, and under flow conditions PECAM distribution was more like patches.

### Podocytes and Endothelial Cells

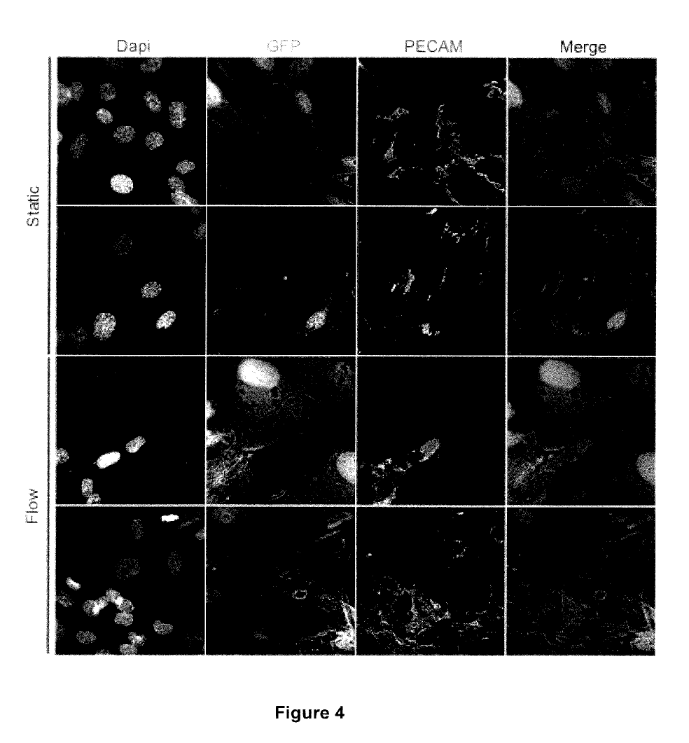

Podocytes were very large under flow conditions, and were a lot smaller under static conditions. The cell types intermingled more under static conditions, which under flow conditions, cell types grouped together more (FIG. 4).

### Example 5: Analysis of Podocytes Under Flow Conditions by Electron Microscopy

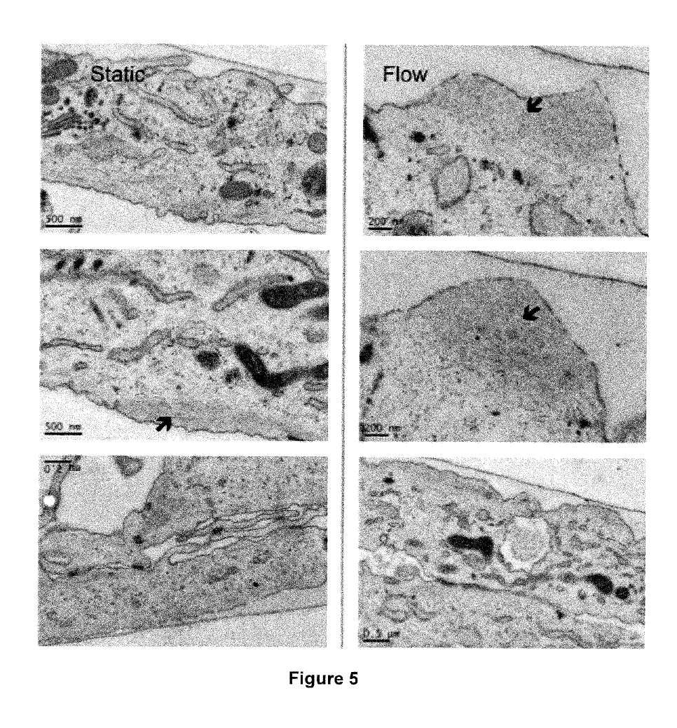

Podocytes having stable expression FLAG-tagged nephrin (passage 18+13) were cultured for 5 days at 37° C., under flow condition or under static conditions, and analysed by transmission electron microscopy (TEM). The cell surface was smoother following culture under static conditions than under flow conditions, and cells cultured under flow conditions had concentrations of actin fibres (FIG. 5 arrows).

### Example 6: Printing of Cell Layers

Cell layers are printed to culture substrates by an inkjet printing method (e.g. see Saunders et al., Delivery of human fibroblast cells by piezoelectric drop-on-demand inkjet printing. Biomaterials 29 (2008) 193-203).

Endothelial cells are deposited in an array pattern on the culture substrate. Following culture of endothelial cells, podocytes are deposited on the endothelial cell layer. Cells are deposited in fixed patterns to form cell sheets of desired shape.

### Example 7—Production of Cell Sheets

### Materials:

Conditionally immortalized glomerular endothelial cells (ciGEnC)
Conditionally immortalized podocytes (glomerular visceral epithelial cells)
Culture flasks (Corning® 75 cm² U-Shaped Canted Neck Cell Culture Flask with Vent Cap)
Endothelial growth medium 2 (EGM2-MV, Cambrex, Wokingham, UK)
RPMI 1640 medium (RPMI 1640 w/L-Glutamine-Bicarbonate, R8758, Sigma)
Fetal bovine serum (heat inactivated, Gibco)
Insulin-Transferrin-Selenium 100×, Gibco, Ref. 41400-045
Temperature-responsive dishes (Nunc® UpCell™ Surface cell culture dish, 3.5 cm culture dish, vented, VWR)
PBS (Dulbecco's phosphate buffered saline, D8537, without calcium chloride and magnesium chloride, Sigma)
Trypsin/EDTA solution (T3924, Sigma)
Falcon tubes (Corning® 50 mL PP Centrifuge Tubes, Conical Bottom with CentriStar™ Cap, Rack Packed, Sterile, Product #430828)
Costar® 6 Well Clear TC-Treated Multiple Well Plates, Individually Wrapped, Sterile, Product #3516)
Coverslips (Coverglass, No 1 thickness, 13 mm diameter, PFM Medical UK ltd)
ACLAR® Embedding Film
4% Paraformaldehyde in phosphate buffered saline
Transmission Electron microscopy (TEM) fixative:
   4% Paraformaldehyde
   2.5% Glutaraldehyde,
   0.1M Hepes
70% Ethanol
Sterile tweezers, pipette tips, stripettes

### Method:

Procedure is performed under sterile conditions unless otherwise stated.
1. Culturing of conditionally immortalized glomerular endothelial cells (ciGEnC):
   Set up 1 culture flask of ciGEnCs (Corning® 75 cm² U-Shaped Canted Neck Cell Culture Flask with Vent Cap) for every 2 temperature-responsive dishes (Nunc® UpCell™ Surface cell culture dish, 3.5 cm culture dish, vented, VWR).
   Cells were cultured in endothelial growth medium 2 (EGM2-MV, Cambrex, Wokingham, UK) containing fetal calf serum (5%) and growth factors as supplied, excepting VEGF.
   Grow cells at a temperature of 33° C. until they become fully confluent. Change endothelial growth medium every 2 to 3 days (10-15 ml medium per 75 cm² culture flask).
2. Culturing of Podocytes:
   Set up 1 culture flask (Corning® 75 cm² U-Shaped Canted Neck Cell Culture Flask with Vent Cap) of podocytes in RPMI 1640 medium (RPMI 1640 w/L-Glutamine-Bicarbonate, R8758, Sigma) supplemented with 10% fetal bovine serum (heat inactivated, Gibco) and 1× Insulin-Transferrin-Selenium (Insulin-Transferrin-Selenium 100×, Gibco, Ref. 41400-045).
   Grow cells at a temperature of 33° C. and change medium every 2 to 3 days.
3. Seeding conditionally immortalized glomerular endothelial cells onto temperature-responsive dishes:
   When ciGEnC reach high confluency (app. 3 000 000 cells/flask), rinse cells 2× with 10 ml of phosphate buffered saline (Dulbecco's phosphate buffered saline, D8537, without calcium chloride and magnesium chloride, Sigma).
   Add 3-5 ml of Trypsin/EDTA solution (T3924, Sigma) and incubate at 37° C. for 5 minutes to detach the cells. Tap flask to help with detachment, add 5-7 ml of supplemented endothelial growth medium 2 to the flask to inactivate the Trypsin.
   Transfer cell suspension to a falcon tube and spin for 4 minutes at 1600 rpm.
   Remove supernatant and resuspend cell pellet in 6 ml of fresh cell culture media. Add 3 ml of the cell suspension to each temperature-responsive dish and incubate at 33° C. until whole layer of cells has formed.
   Change media every 2-3 days. Usually it takes about a week for the cells to become confluent enough to yield a cell sheet.
4. Seeding conditionally immortalized podocytes onto coverslips and ACLAR® Film:
   Split one confluent 75 cm² culture flask with conditionally immortalized podocytes (approx. 2.5 million cells/flask) onto glass coverslips and ACLAR® Film in a 6-well cell culture plate (on the same day as you seed the ciGEnC onto temperature-responsive dishes).
   Rinse cells once with 10 ml of phosphate buffered saline (Dulbecco's phosphate buffered saline, D8537, without calcium chloride and magnesium chloride, Sigma).
   Add 3-5 ml of Trypsin/EDTA solution (T3924, Sigma) and incubate at 37° C. for 5 minutes to detach the cells. Tap flask to help with detachment, add 5-7 ml supplemented RPMI 2 medium to the flask to inactivate the Trypsin.
   Transfer cell suspension to a falcon tube and spin for 4 minutes at 1600 rpm.
   Remove supernatant and resuspend cell pellet in 18 ml of fresh cell culture media.
   Prepare 6 well culture dish:
      Cut out 3 squares from the ACLAR® Film (need to fit inside well of the culture plate)
      Sterilize in 70% Ethanol
      Sterilize 3 glass cover slips in 70% Ethanol
      Rinse in phosphate buffered saline
      Add each square/coverslip into one well of the culture plate
   Add 3 ml of podocyte cell suspension to each well of the culture plate.
   Incubate at 33° C., change RPMI 1640 medium every 2-3 days.
5. Harvesting cell sheets from temperature-responsive dishes:
   When ciGEnCs on temperature-responsive dish reach high confluency state, move dish to room temperature, remove medium and add PVDF membrane (part of the kit) with sterile tweezers (sterilize with 70% Ethanol before use).
   Add couple of drops of cold medium (cells don't try out and PVDF doesn't float off), leave for a minimum of 30 min at room temperature
   Loosen edges with sterile tweezers and pull cell sheet towards PVDF membrane, check under microscope to see if cell sheet has loosened (leave longer if not). Grab PVDF with tweezers and lift up both, cell sheet and membrane from dish. Check dish under microscope if cells are gone.
   Remove medium from podocytes grown on cover slips and ACLAR® Film inside a 6-well culture plate. Podocytes reach very high confluency at that point.
   Cut cell sheet and membrane with scissors in two halves (cell sheets can be left in one piece if preferred). Put each half onto the podocytes. Add small amount of endothelial medium onto cell sheet to avoid cells drying out.
   Incubate at 33° C. for 30 min to let cell sheet attach to podocytes, add more endothelial medium and let PVDF membrane come off on its own. Incubate dishes at 37° C. for 14 days. Change media regularly every 2 to 3 days.
6. Fixation of cells:
   Fix glass cover slips with 4% Paraformaldehyde solution for 15 min. Remove fixative and add PBS. Store in fridge. Samples are ready for Immunocytochemistry staining.
   ACLAR®: Don't remove medium, add as much TEM fixative as medium to wells and leave in fix, never let ACLAR® dry out. Store in fridge until samples can be processed for Transmission Electron Microscopy.

1. A multilayer sheet of cells, comprising a layer of endothelial cells, and an adjacent layer of podocytes.

2. The multilayer sheet of cells according to claim 1, wherein the endothelial cells are glomerular endothelial cells.

3. The multilayer sheet of cells according to claim 1 or claim 2, further comprising an extracellular matrix disposed between the cell layers.

4. The multilayer sheet of cells according to any one of claims 1 to 3, wherein the multilayer sheet of cells is provided on a support surface.

5. The multilayer sheet of cells according to any one of claims 1 to 4, wherein the multilayer sheet of cells is detachable from the support surface without enzymatic treatment, optionally wherein the support surface is a temperature responsive support surface.

6. The multilayer sheet of cells according to any one of claims 1 to 5, wherein the cell layers are confluent cell layers.

7. The multilayer sheet of cells according to any one of claims 1 to 6, which is tolerant of flow conditions.

8. The multilayer sheet of cells according to any one of claims 1 to 6, which is impervious to proteins larger than about 100 kDa.

9. An in vitro cell culture, comprising a layer of endothelial cells, and an adjacent layer of podocytes, optionally wherein the endothelial cells are glomerular endothelial cells.

10. An in vitro cell culture according to claim 9, comprising an extracellular matrix disposed between the cell layers.

11. An in vitro cell culture according to claim 9 or claim 10, provided on a support surface.

12. An in vitro cell culture according to claim 11, wherein the support surface is a surface from which the multilayer sheet of cells is detachable without enzymatic treatment, optionally wherein the support surface is a temperature responsive support surface.

13. An in vitro cell culture according to any one of claims 9 to 12, wherein the cell layers are confluent cell layers.

14. A device, having two compartments separated by a multilayer sheet of cells according to any one of claims 1 to 8.

15. The device according to claim 15, additionally comprising a liquid/fluid permeable support for the multilayer sheet of cells.

16. The device according to claim 14 or claim 15, comprising a liquid/fluid inlet to the compartment adjacent the layer of endothelial cells, and a liquid/fluid outlet from the compartment adjacent the layer of podocytes.

17. The device according to claim any one of claims 14 to 16, additionally comprising a compartment comprising renal epithelial cells.

18. The device according to claim any one of claims 14 to 17, wherein the compartment comprising renal epithelial cells is in liquid/fluid communication with the compartment adjacent the layer of podocytes.

19. Use of a multilayer sheet of cells according to any one of claims 1 to 8, or of a device according to any one of claims 14 to 18, in an in vitro assay.

20. The use according to claim 19, wherein the assay is a toxicity assay.

21. The use according to claim 19 or claim 20, wherein the assay measures transfer of a substance across the multilayer sheet of cells.

22. The use according to any one of claims 19 to 21, wherein the assay measures a property of the multilayer sheet of cells, or a property of a component of the multilayer sheet of cells.

23. An artificial kidney, comprising a multilayer sheet of cells according to any one of claims 1 to 8, or a device according to any one of claims 14 to 18.

24. A dialysis apparatus, comprising a multilayer sheet of cells according to any one of claims 1 to 8, or a device according to any one of claims 14 to 18.

25. Use of a multilayer sheet of cells according to any one of claims 1 to 8, a device according to any one of claims 14 to 18, an artificial kidney according to claim 23, or a dialysis apparatus according to claim 24, to filter blood or plasma.

26. The multilayer sheet of cells according to any one of claims 1 to 8, device according to any one of claims 14 to 18, artificial kidney according to claim 23, or dialysis apparatus according to claim 24, for use in a method of treatment by dialysis.

27. A method of treating a patient in need of dialysis, the method comprising use of a multilayer sheet of cells according to any one of claims 1 to 8, a device according to any one of claims 14 to 18, an artificial kidney according to claim 23, or a dialysis apparatus according to claim 24, to dialyse blood or plasma.

28. A method for producing a multilayer sheet of cells, the method comprising:
(i) providing a layer of endothelial cells;
(ii) providing a layer of podocytes; and
(iii) contacting the layers of cells and culturing the contacted cell layers in vitro;
thereby producing a multilayer sheet of cells.

29. A method for producing a multilayer sheet of cells, the method comprising:
(i) providing a layer of endothelial cells, optionally glomerular endothelial cells;
(ii) layering podocytes onto the layer of endothelial cells; and
(iii) culturing the cells in vitro;
thereby producing a multilayer sheet of cells.

30. The method according to claim 29, wherein step (i) comprises contacting endothelial cells or glomerular endothelial cells with a support surface, and culturing the cells on the support surface in vitro.

31. The method according to claim 30, wherein the support surface is a surface from which the multilayer sheet of cells is detachable without enzymatic treatment, optionally wherein the support surface is a temperature responsive support surface.

32. The method according to any one of claims 29 to 31, wherein the method additionally comprises:
(iv) detaching the multilayer sheet of cells from the support surface.

33. A method for producing a multilayer sheet of cells, the method comprising:
(i) providing a layer of podocyte cells;
(ii) providing a layer of endothelial cells, optionally glomerular endothelial cells,
(iii) placing the layer of endothelial cells onto the layer of podocyte cells, or placing the layer of podocyte cells onto the layer of endothelial cells; and
(iii) culturing the cells in vitro;
thereby producing a multilayer sheet of cells.

34. The method according to claim 33, wherein one or both of the layer of podocyte cells of (i) and layer of endothelial cells of (ii) is a layer on a support surface from which the cell layer is detachable without enzymatic treatment, optionally wherein the support surface is a temperature responsive support surface.

35. The method according to claim 33 or 34, wherein the method additionally comprises detaching the multilayer sheet of cells from the support surface.

36. A bilayer sheet of cells, comprising a layer of endothelial cells, and a layer of podocytes.

37. A bilayer sheet of cells consisting of a layer of endothelial cells and a layer of podocytes.

38. A bilayer sheet of cells consisting of a layer of endothelial cells, a layer of podocytes and extracellular matrix disposed between the cell layers.
